(19) United States
(12) Patent Application Publication
(10) Pub. No.: US 2007/0197970 A1
(43) Pub. Date: Aug. 23, 2007

(54) INTRAFASCIAL CATHETER INTRODUCER SYSTEM AND METHOD OF DELIVERY OF POSTOPERATIVE ANESTHETICS

(76) Inventor: Jane Shen-Gunther, Dupont, WA (US)

Correspondence Address: GREENBERG TRAURIG, 3773 HOWARD HUGHES PARKWAY, SUITE 500 NORTH, LAS VEGAS, NV, 89169, US

(21) Appl. No.: 11/466,401

(22) Filed: Aug. 22, 2006

Related U.S. Application Data
(60) Provisional application No. 60/712,609, filed on Aug. 22, 2005.

(51) Int. Cl. A61M 5/178

(52) U.S. Cl. 604/164.01

(57) ABSTRACT

A surgical procedure for managing postoperative pain is disclosed. More particularly, the technique involves use of intrafascial catheter introducer to thread a soaker catheter or the like through fascial sheaths of the human body (e.g., abdominal and chest walls) to relieve postoperative pain. The use of the soaker catheter in apposition to the intercostal nerves enhances the efficacy of the soaker catheter that is normally laid in the subcutaneous space. Case study results evidence the enhanced efficacy of the surgical procedure.

### CROSS REFERENCE TO RELATED APPLICATION

This application claims the benefit of provisional patent application No. 60/712,609 filed Aug. 22, 2005.

### FIELD OF THE INVENTION

The embodiments of the present invention relate to the use of intrafascial catheter introducer to thread a soaker catheter or the like through fascial sheaths of the human body (e.g., abdominal and chest walls) to relieve postoperative pain.

### BACKGROUND

An estimated 28 million operations are performed each year in. the United States alone. Management of postoperative pain has not changed for hundreds of years and remains a challenge for surgeons and doctors. The current modalities of analgesia include systematic opioids, nonsteroidal anti-flammatory drugs (NSAIDS), cyclooxygenase-2 (COX-2) inhibitors, and epidural local anesthetics. Despite these modern therapies, postoperative pain control remains problematic.

For centuries, opioids have served as the principal remedy for pain. Indeed, the use of opioids dates back to the third century B.C. and continues to this day. The discovery and isolation of cocaine led to the development of modern local anesthetics for pain management. In 1950, Blades and Ford, disenchanted with the undesirable and suboptimal effects of morphine for postoperative analgesia, improvised a new method for thoracic surgery. The method involved the use of small caliber tubes positioned directly against the intercostal muscles that would penetrate the skin to allow injections of procaine postoperatively. Despite proven efficacy, the technique was not widely adopted.

In 1999, I-Flow Corporation of Lake Forest, Calif. introduced its ON-Q® anesthetic system to the field of surgery. The ON-Q system is a continuous local anesthetic wound irrigation system that delivers an anesthetic through a soaker catheter laid in the subcutaneous space. Since its introduction, the device's efficacy has been the subject of multiple publications. However, the current placement of the catheter in the subcutaneous space is not ideal. Although not taught or suggested in any published literature, the inventors hereof determined, as evidenced by the case studies detailed below, that greater efficacy and analgesia can be realized by placing the catheter in apposition to intercostal nerves.

Thus, there exists a need for a device and method for intrafascial placement of a soaker catheter or the like to more effectively manage postoperative pain.

### SUMMARY

Accordingly, a surgical procedure embodiment of the present invention comprises: prior to closing an incision, positioning a soaker catheter in apposition to intercostal nerves in or near a chest or abdominal wall. Another surgical procedure embodiment of the present invention comprises: threading the intrafascial catheter introducer (ICI) through the right or left rectus sheath from the fascial edge below the arcuate line to the superior fascial apex; placing an introducer needle through the skin just beyond the apex of the incision; feeding the tip of the catheter through the distal hole of the intrafascial catheter introducer (ICI); placing the catheter at a desired location in the fascial incision; and withdrawing the intrafascial catheter introducer (ICI) such that the soaker catheter remains threaded through the rectus sheath. This may be performed similarly for transverse thoracic or abdominal incisions.

Other variations, embodiments and features of the present invention will become evident from the following detailed description, drawings and claims.

### BRIEF DESCRIPTION OF THE DRAWINGS

FIG. 1 illustrates a conventional Pneumoneedle insufflation needle;

FIG. 1a illustrates a surgical procedure whereby a Verres® needle is typically used;

FIG. 2 illustrates an ON-Q system;

FIGS. 3a-3c illustrate various views of a intrafascial catheter introducer used for facilitating the embodiments of the present invention;

FIGS. 4a-4c illustrate a series of diagrams showing intrafascial catheter placement using a Pneumoneedle® as used in experimental practice;

FIG. 5 illustrates a patient characteristics table;

FIG. 6 illustrates a diagnosis table

FIG. 7 illustrates a surgical data table

FIG. 8 illustrates a longevity and complication rates table; and

FIG. 9 illustrates a postoperative pain scores table.

### DETAILED DESCRIPTION

It will be appreciated by those of ordinary skill in the art that the invention can be embodied in other specific forms without departing from the spirit or essential character thereof. The presently disclosed embodiments are therefore considered in all respects to be illustrative and not restrictive.

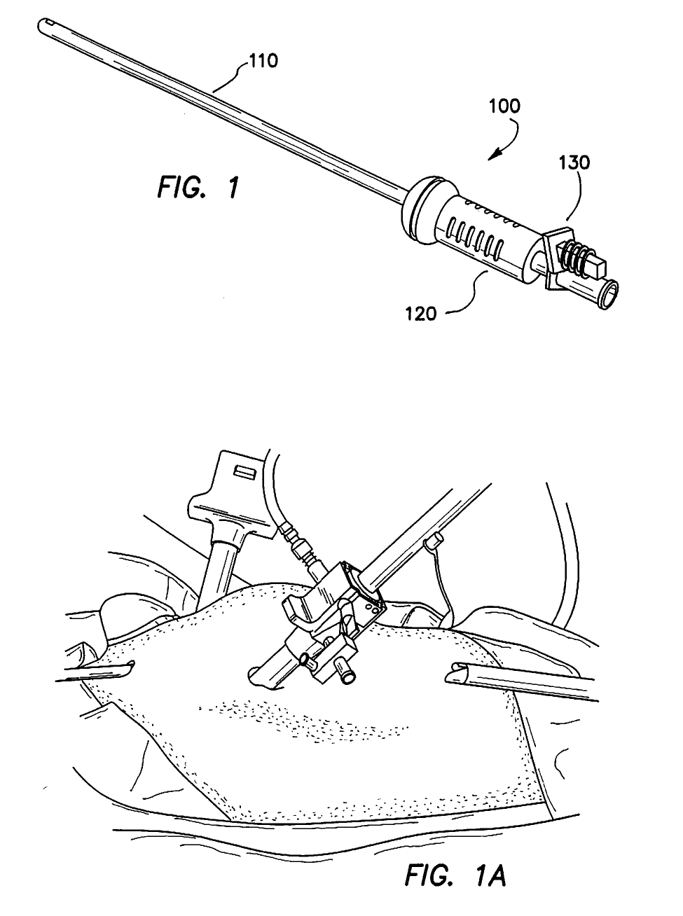

Initial reference is made to FIG. 1 illustrating a perspective view of a conventional Pneumoneedle insufflation needle generally identified by reference numeral 100. The Pneumoneedle insufflation needle 100 comprises a spring-loaded, blunt-stylet mechanism similar in function to a Veress needle. It is used to establish pneumoperitoneum prior to abdominal endoscopy. A stainless steel needle 110 is attached at its proximal end to a plastic handle 120. The handle may be cylindrical in shape for comfort in a user's hand and contains a stopcock and luer lock connector 130 for insufflating the abdominal cavity. As shown in FIG. 1a, the Verres needle (not shown here) is normally inserted through the umbilicus into the abdominal cavity for inflation of carbon dioxide to elevate the abdominal wall. Afterwards, pelvic surgery can then be conducted through the larger trocar sheaths as demonstrated in FIG. 1a.

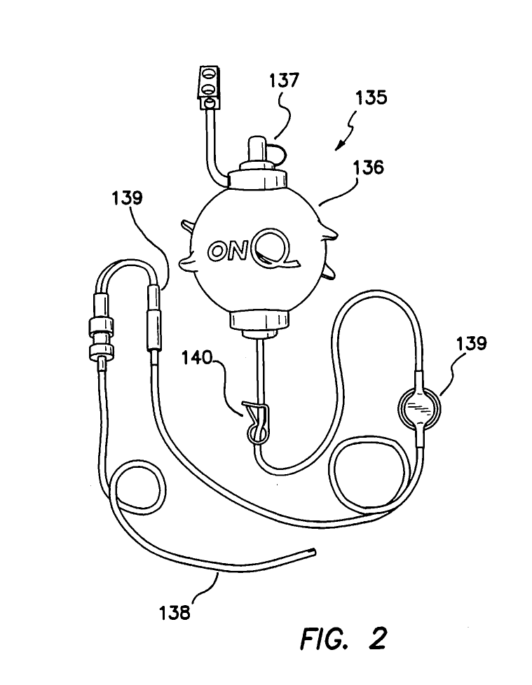

FIG. 2 illustrates an ON-Q pain management system 135. The system 135 comprises a pump 136, including a fill port 137, and catheter 138. A flow restrictor 139 controls the rate of flow of the medication from the pump 136 and a filter 139 ensures that the medication is sterile when it reaches the patient. A clamp 140 provide a means for quickly stopping the flow of medication. Conventionally, the catheter 138 is placed in the subcutaneous space after surgery.

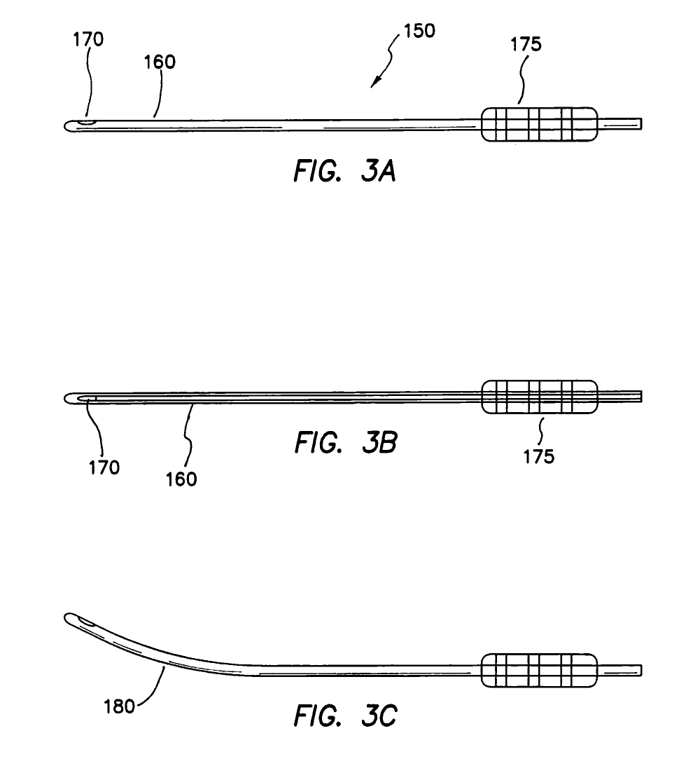

FIGS. 3a-3b show various views, namely a side view and top view, respectively, of a intrafascial catheter introducer (ICI), having a straight tip, for facilitating the embodiments of the present invention generally identified by reference numeral 150. The introducer 150 comprises an elongated, hollow needle or shaft 160 having a portal opening 170 at one end thereof. A ribbed handle 175 is positioned adjacent to a second end thereof. The handle 170 provides a means for controlling the introducer 150 and is ideally cylindrical in shape to accommodate a user's hand. FIG. 3c shows a intrafascial catheter introducer 150 having a curved tip 180. In one specific embodiment, the ICI comprises a blunt tipped, slender stainless steel hollow tube with the following dimensions (14 gauge diameter, 120 or 150 mm length). A ribbed plastic handle is attached at the proximal end for easy handling. A small (2×3 mm) portal is located at the distal tip to allow threading of small caliber infusion catheters to be placed at strategic intrafascial locations of the body. After threading the ON-Q catheter, the introducer is simply removed with the catheter left in situ. The straight introducer is designed for vertical abdominal or chest wall incisions; whereas, the curved introducer is for transverse incisions, i.e. c-section “bikini type” incisions.

Management of postoperative pain has always been a daunting challenge for surgeons. For example, abdominal surgeries requiring incisions into the upper portion of the abdomen are well recognized for inducement of severe postoperative pain. Consequently, patients undergoing such surgeries may experience splinting of accessory muscles of respiration, loss of sighing and reduction in the vital capacity that can individually or collectively lead to significant pulmonary morbidity. Other adverse effects of inadequate analgesia include cardiovascular, thromboembolic and gastrointestinal complications.

In gynecologic oncology, full abdominal exposure through an extended incision is often necessary for surgical staging and cytoreduction. An effective means of managing postoperative pain is critical to the welfare of such patients. The current modalities of analgesia include systemic opioids, nonsteroidal anti-flammatory drugs (NSAIDs), cyclooxygenase-2 (COX-2) inhibitors and epidural local anesthetics. Despite the modern therapies, postoperative pain control remains suboptimal.

One offered solution to postoperative pain control is the above-referenced ON-Q system that acts as a continuous local anesthetic wound irrigation system delivering an anesthetic through a soaker catheter laid in the subcutaneous space. Unfortunately, positioning the ON-Q in the subcutaneous space is not the most effective solution to managing postoperative pain. Accordingly, the embodiments of the present invention involve placement of a soaker catheter in apposition to intercostal nerves.

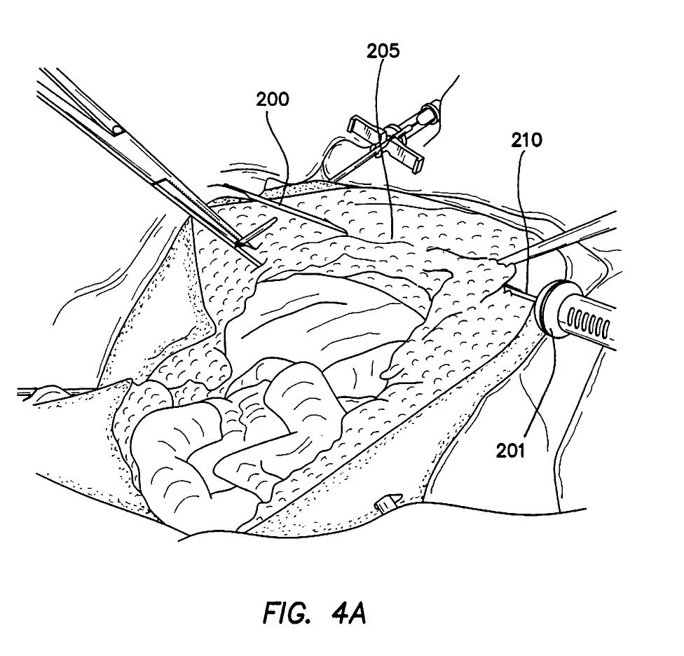

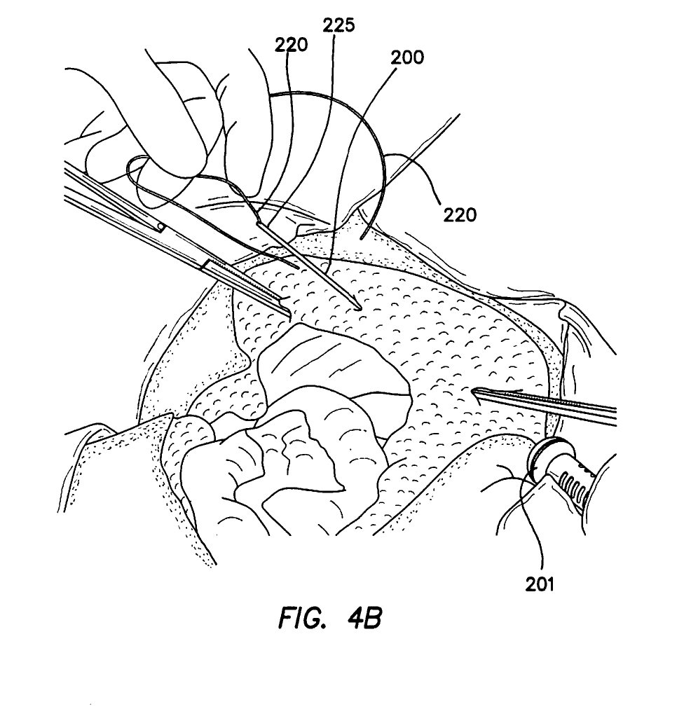

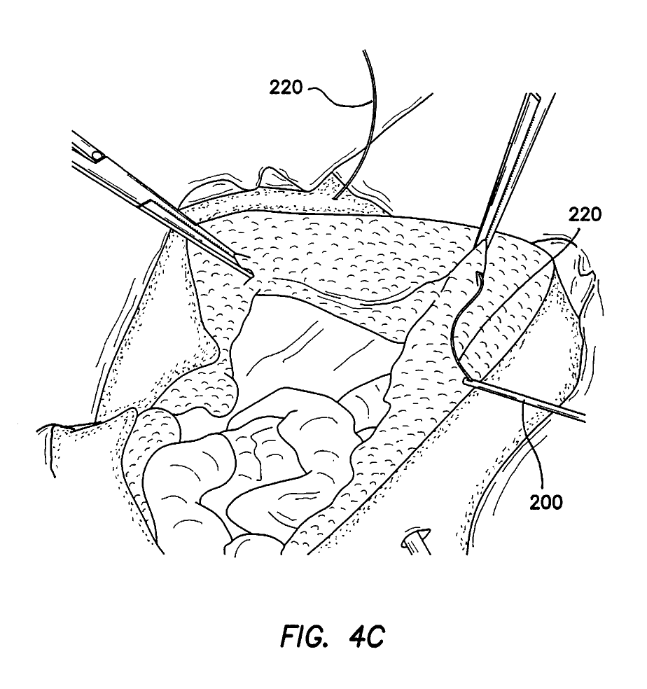

FIGS. 4a-4c show placement of the soaker catheter in apposition to the intercostal nerves during an abdominal type surgery using a Verres needle 200 rather than the ICI. The patient underwent routine preoperative bowel preparation, prophylactic intravenous antibiotics, and thrombosis prophylaxis with sequential compression stockings. A standard midline vertical laparotomy incision is made. Incisions may be extended above the umbilicus as necessary for exposure. At the completion of the operation, prior to fascial closure, the following technique, as detailed in FIGS. 4a-4c was utilized. First, as shown in FIG. 4a, a Verres needle 200 (Ethicon EndoSurgery, Cincinnati, Ohio) having handle 201 is threaded through the right or left rectus sheath 205 from the fascial edge 210 below the arcuate line to the superior fascial apex 215. Next, the Verres needle 200 is introduced through the skin approximately 3 cm from the edge of the incision at the apex. As shown in FIGS. 4b and 4c, the tip of the catheter 220 is fed into the distal tip/hole 225 of the Verres needle 200. A Verres needle 200 spring is then gently pulled back with just enough force to grasp the catheter tip. Undue or excessive or complete retraction of the Verres needle 200 is to be avoided since it tends to shear off the tip of the catheter 220. As shown in FIG. 4c, the Verres needle 200 is then withdrawn in order to deliver and place the distal tip of the catheter 220 at the midpoint of the fascial incision. Although the previous figures show the use of a Verres needle 200, in practice such use is difficult because the tip of the Verres needle 200 is sharp and excessive retration may shear off the tip of the ON-Q catheter. Therefore, the embodiments of the present invention utilize the ICI in place of the Verres needle 200. The wound is then closed using a mass closure technique employing a looped O-PDS suture. The catheter 220 is maintained within the fascial sheath encircled by the PDS suture placed 1 to 1.5 cm from the fascial edge. Subcutaneous closure is not used. A 10 mm Jackson-Pratt drain is placed in the subcutaneous tissue for closed-suction drainage. The ON-Q soaker catheter 220 is connected to a bupivacaine (0.5%) filled elastomer pump and secured to the skin with transparent adhesive dressing. The predetermined flow rate of bupivacaine is 2 mL/hr with an infusion segment of 6.5 cm. The predetermined total fill volume of the elastomer pump is 270 mL which permits over 120 hours (or five days) of drug delivery time.

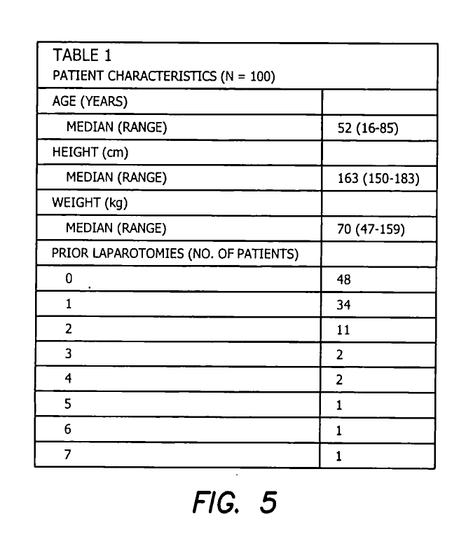

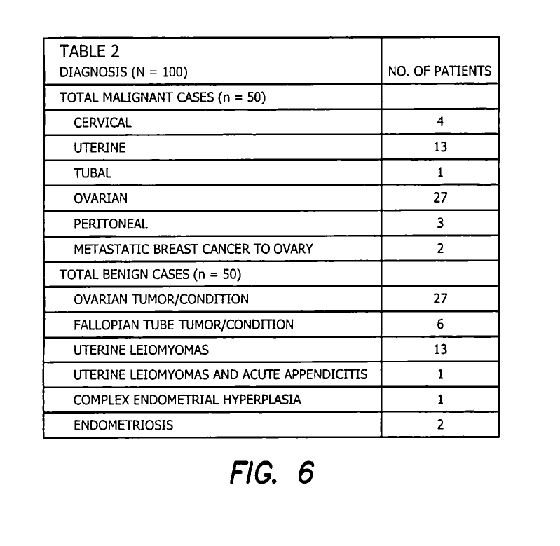

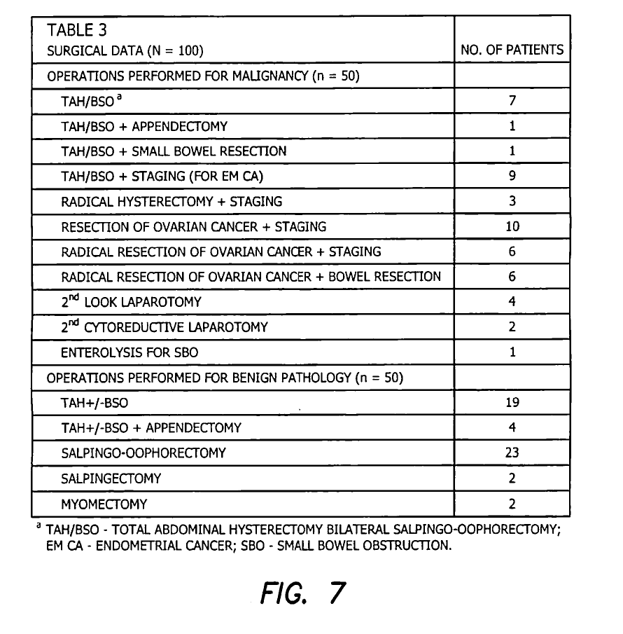

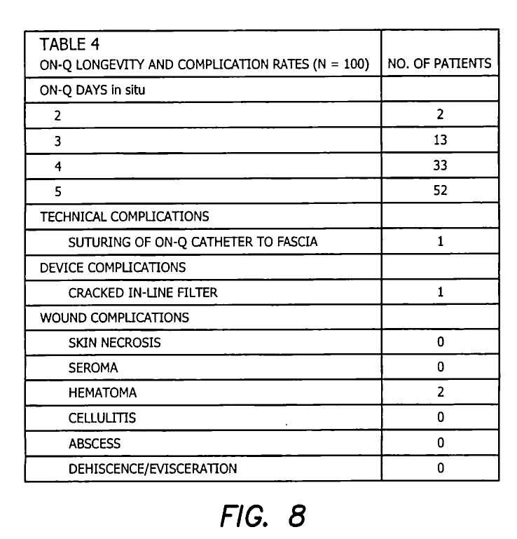

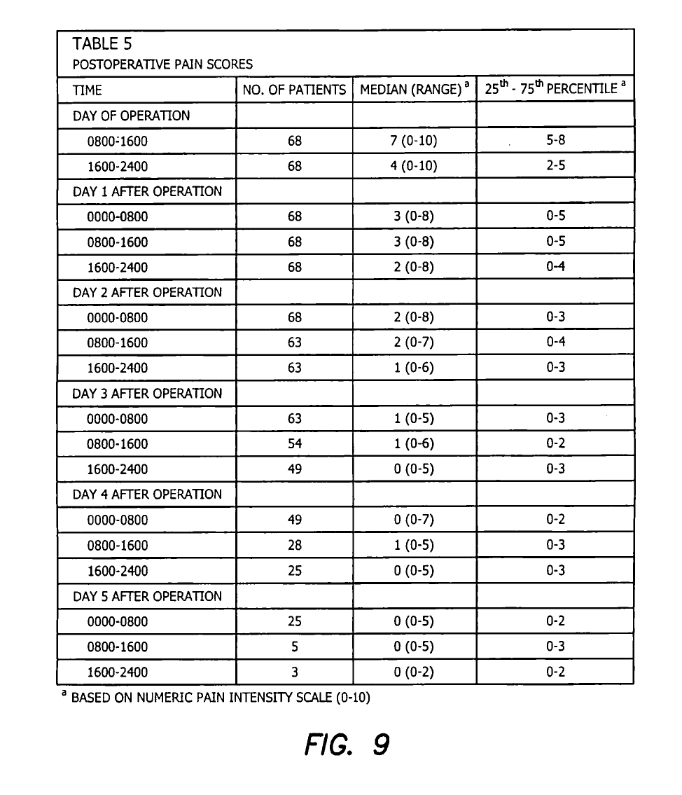

The procedure described and shown in FIGS. 4a-4c was conducted on 100 patients during a confidential case study period. Patient characteristics including age, height and weight are summarized in Table I (FIG. 5). 48 patients had no prior abdominal surgeries, 34 patients had 1 and 18 patients had 2 or more laparotomies. Table II (FIG. 6) lists the pathologic diagnosis of the patients. 50 operations were performed for gynecologic malignancies with the most common indication being ovarian carcinoma. An additional 50 operations were performed for benign gynecologic conditions. All surgical procedures performed are listed in Table III (FIG. 7). 54 patients required an extended (supraumbilical) incision and 46 received a standard length (subumbilical) incision. As listed in Table IV (FIG. 8), the majority (85%) of the ON-Q systems were left in situ for at least 4 or 5 days. Four patients were discharged earlier than usual since their incisional pain was well-controlled with the ON-Q pump. The median pain scores of the subjects starting from DOS to POD 5 are presented in Table V (FIG. 9).

In the case study referenced above, the ON-Q anesthetic system was positioned in apposition to the intercostal nerves and used as an adjunct to postoperative pain management. Studies on somatic neuroanatomy and peripheral nociception provided the rationale for the novel placement technique. Based on the classical anatomic description of the rectus abdominus muscle, the muscle is innervated by the terminal branches of the lower six intercostal nerves. The intercostal nerves traverse posteriorly to the internal oblique muscle to terminate into the lateral cutaneous and medial musculocutaneous branches. As for the origin of incisional pain, in vertical laparotomies, the linea alba is incised either with cautery or sharp instruments and closed under mechanical tension with sutures. Studies of peripheral neueral mechanisms of pain indicate that nociceptors are excited by injurious stimuli such as heat, mechanical or chemical stimuli, and inflammation. Therefore, the process of fascial incision and closure appears to play a critical role in the genesis of pain. Nociception derived from the skin, subcutaneous adipose and peritoneum is believed to be inconsequential. Based on the above, anesthetic perfusion along the fascial incision-suture line with simultaneous permeation of the intercostal nerves (medial branch) provides maximum benefit.

The analgesic performance results of the study revealed a precipitous drop in median pain score from 7 in the post-anesthesia care unit (PACU) to 4 on the evening shift of day-of-surgery. The pain score continued its gradual decline from 3 to 0 over the next four postoperative days.

Although the invention has been described in detail with reference to several embodiments, additional variations and modifications exist within the scope and spirit of the invention as described and defined in the following claims.

I claim:

1. A surgical procedure comprising:
prior to closing an incision, positioning a soaker catheter in apposition to intercostal nerves in or near a chest or abdominal wall.

2. The surgical procedure of claim 1 further comprising utilizing an intrafascial catheter introducer, comprising a hollow needle having an opening at one end and a handle at an opposite end, to position the soaker catheter in apposition to intercostal nerves in or near a chest or abdominal wall.

3. A surgical procedure comprising:
making an incision near an abdominal or chest wall; and
prior to closing an incision, positioning a soaker catheter in apposition to intercostal nerves in or near a chest or abdominal wall wherein the soaker catheter is positioned utilizing an intrafascial catheter introducer comprising a hollow needle having an opening at one end and a handle at an opposite end.

4. A surgical procedure comprising:
threading the intrafascial catheter introducer (ICI) through the right or left rectus sheath from the fascial edge below the arcuate line to the superior fascial apex;
placing an introducer needle through the skin just beyond the apex of the incision;
feeding the tip of the catheter through the distal hole of the intrafascial catheter introducer (ICI);
placing the catheter at a desired location in the fascial incision;
and withdrawing the intrafascial catheter introducer (ICI) such that the soaker catheter remains threaded through the rectus sheath.

5. The surgical procedure of claim 4 wherein the procedure is performed for transverse thoracic or abdominal incisions.

6. A medical instrument comprising a blunt tip hollow tube with a ribbed handle at one end thereof and a portal located at a second end thereof wherein said portal and hollow tube are configured to receive a catheter.

7. The medical instrument of claim 6 wherein the hollow tube is between 120 to 150 mm in length and has a diameter of approximately 14 mm, and said portal is approximately 2×3mm.

8. The medical instrument of claim 6 wherein the hollow tube is fabricated of stainless steel.
